(19) United States
(12) Patent Application Publication
(10) Pub. No.: US 2017/0361040 A1
(43) Pub. Date: Dec. 21, 2017

(54) Aromatherapy Vaporization Device

(71) Applicant: MICHAEL ALEXANDER TRZECIESKI, MID LEVELS (HK)

(72) Inventor: MICHAEL ALEXANDER TRZECIESKI, MID LEVELS (HK)

(21) Appl. No.: 15/626,656

(22) Filed: Jun. 19, 2017

Related U.S. Application Data
(60) Provisional application No. 62/352,491, filed on Jun. 20, 2016.

(51) Int. Cl. A61M 15/06; A24F 47/00; A61M 11/04; A24F 1/30

(52) U.S. Cl. CPC A61M 15/06; A24F 1/30; A24F 47/008; A61M 11/042; A61M 2205/3653; A61M 2205/8262; A61M 2209/08; A61M 2205/0216

(57) ABSTRACT

A phyto material vaporization device is disclosed for being powered by an atomizer power supply having a central axis and a control button for being attached to an aromatherapy water pipe having an input port in fluid communication with an inhalation aperture. A water pipe engagement member is provided for being releasably coupled with the aromatherapy water pipe and a fastening mechanism for releasably fastening the water pipe engagement member to the aromatherapy water pipe. An atomizer power supply receptacle for releasably coupling with the atomizer power supply for receiving of electrical energy therefrom and a conduction heating element for receiving of the phyto material and for being disposed proximate the aromatherapy water pipe input port for restricting of ambient air flowing into the input port and having an electrical connection for electrically coupling with the atomizer power supply receptacle.

### CROSS REFERENCE TO RELATED APPLICATION

This application benefits from the priority of U.S. Provisional Applications 62/352,491 filed on Jun. 20, 2016, which is incorporated herein by reference.

### TECHNICAL FIELD OF THE INVENTION

The technical field relates to a device and method that provides for vaporization of phyto materials.

### BACKGROUND OF THE INVENTION

Prior art vaporizers utilize a battery module that is coupled with an atomizer in order to heat liquid or phyto material to a predetermined temperature in order to release vapor.

These battery modules are typically coupled with the atomizer using a 510 internal thread or eGo external thread, as is known in the art. Other coupling mechanisms have also been used in the industry, but these two are the standard.

The atomizer battery usually consists of a charging port, in some cases this port is the 510 internal threaded port, and a control button as well as a control circuit. The 510 threaded port or the eGo external thread work to provide electrical power to the atomizer. The control button is used in order to provide electrical power from the atomizer to the 510 or eGo thread.

Many of the existing devices that are in the market require a torch to heat up a glass or metal or ceramic piece and to get it hot so that phyto material or phyto material extract is placed on it so that when its hot enough, 500 F to 700 F, the phyto material vaporizers. Unfortunately, this requires a torch to heat up the glass or metal or ceramic piece and as such its not convenient and also can be a fire hazard. Heating up to such high temperatures without any control can lead to combusting and not vaporization which is not as harmful.

It is therefore an object of the invention to provide an aromatherapy vaporization device that overcomes the aforementioned deficiencies.

### SUMMARY

In accordance with the embodiments of the invention there is provided a phyto material vaporization device for being powered by an atomizer power supply having a central axis and a control button for being attached to an aromatherapy water pipe having an input port in fluid communication with an inhalation aperture comprising: a water pipe engagement member for being releasably coupled with the aromatherapy water pipe; a fastening mechanism for releasably fastening the water pipe engagement member to the aromatherapy water pipe; an atomizer power supply receptacle for releasably coupling with the atomizer power supply for receiving of electrical energy therefrom; a conduction heating element comprising first and second electrical contacts and, conduction heating element for receiving of the phyto material and for being disposed proximate the aromatherapy water pipe input port for restricting of ambient air flowing into the input port and having an electrical connection for electrically coupling with the atomizer power supply receptacle, wherein, upon coupling of the atomizer power supply with the atomizer power supply receptacle and by actuating of the control button, electrical energy from the atomizer power supply flows to the conduction heating element for heating of the phyto material for forming phyto material vapor and for the formed phyto material vapor and ambient air to flow into the input port and past the conduction heating element and parallel to the first and second electrical contacts and for subsequent inhalation from the inhalation aperture by the user.

In accordance with the embodiments of the invention there is provided a phyto material vaporization device for being powered by an atomizer power supply having a central axis and a control button for being attached to an aromatherapy water pipe having an input port in fluid communication with an inhalation aperture comprising: a water pipe engagement member for being releasably coupled with the aromatherapy water pipe; a fastening mechanism for releasably fastening the water pipe engagement member to the aromatherapy water pipe; an atomizer power supply receptacle for releasably coupling with the atomizer power supply for receiving of electrical energy therefrom, the atomizer power supply receptacle for being frictionally engaged within the water pipe engagement member for allowing of rotation of the atomizer power supply receptacle within the water pipe engagement member about a central axis of the atomizer power supply; a conduction heating element for receiving of the phyto material and for being disposed proximate the aromatherapy water pipe input port having an electrical connection for electrically coupling with the atomizer power supply receptacle, wherein, upon coupling of the atomizer power supply with the atomizer power supply receptacle and by pushing of the control button, electrical energy from the atomizer power supply flows to the conduction heating element for heating of the phyto material for forming phyto material vapor and for the formed phyto material vapor and ambient air to flow into the input port and past the conduction heating element upon inhalation from the inhalation aperture by the user.

In accordance with the embodiments of the invention there is provided a phyto material vaporization device for being powered by an atomizer power supply having a central axis and a control button for being attached to an aromatherapy water pipe having an input port in fluid communication with an inhalation aperture comprising: a water pipe engagement member for being releasably coupled with the aromatherapy water pipe; a fastening mechanism for releasably fastening the water pipe engagement member to the aromatherapy water pipe; an atomizer power supply receptacle for releasably coupling with the atomizer power supply for receiving of electrical energy therefrom; a conduction heating element for receiving of the phyto material and for being disposed proximate the aromatherapy water pipe input port for restricting of ambient air flowing into the input port and having an electrical connection for electrically coupling with the atomizer power supply receptacle, wherein, upon coupling of the atomizer power supply with the atomizer power supply receptacle and by actuating of the control button, electrical energy from the atomizer power supply flows to the conduction heating element for heating of the phyto material for forming phyto material vapor and for the formed phyto material vapor and ambient air to flow into the input port and past the conduction heating element upon inhalation from the inhalation aperture by the user.

### BRIEF DESCRIPTION OF THE DRAWINGS

FIG. 1A illustrates a Prior Art atomizer power supply;

FIG. 1B illustrates the power output port of the prior art atomizer power supply;

FIG. 1C illustrates a prior art aromatherapy water pipe;

FIG. 2A illustrates the preferred embodiment of the invention, a phyto material vaporization device without the prior art water pipe and without the prior art atomizer power supply;

FIG. 2B illustrates air and vapor flow past the conduction heating element;

FIG. 2C illustrates the preferred embodiment of the invention 100 coupled to a water pipe but does not show the an atomizer power supply;

FIG. 2D illustrates the preferred embodiment of the invention coupled to a water pipe with the atomizer power supply;

FIG. 3A illustrates the atomizer power supply receptacle being rotated about the central axis of the atomizer power supply within the water pipe engagement member for allowing of rotation of the atomizer power supply;

FIG. 3B illustrates the atomizer power supply receptacle in its preferred orientation about the central axis of the atomizer power supply.

### DETAILED DESCRIPTION OF EMBODIMENTS OF THE INVENTION

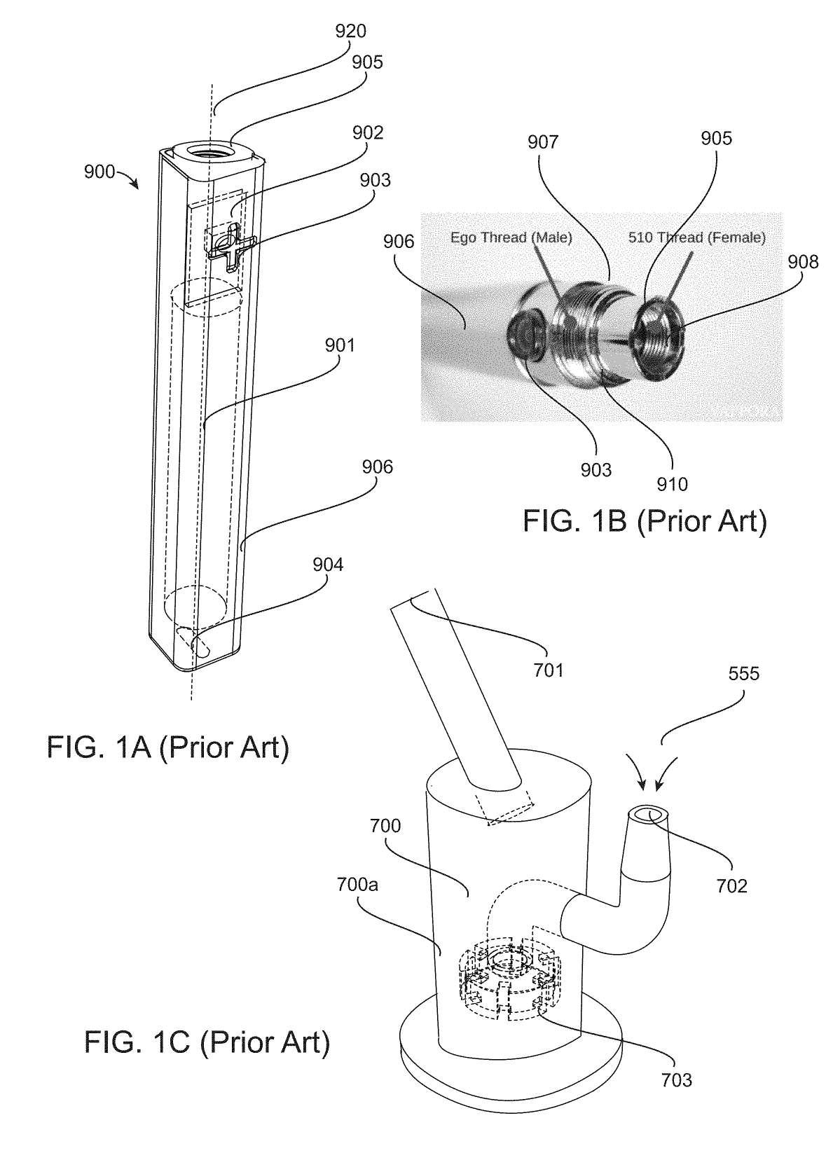

FIG. 1A illustrates a Prior Art atomizer power supply 900 having an internal lithium battery 901 disposed within a housing 906 coupled with a control circuit 902 and a charging port 904. The control circuit 902 is electrically coupled with the control button 903. A power output port 905 is coupled with a prior art atomizer (not shown) for delivering of electrical power thereto. A central axis 920 of the atomizer power supply 900 is shown. Referring to FIG. 1B, the power output port 905 is shown having the eGo external male thread 910 and the 510 female thread 908. The 510 female thread is more common and both are well known in the art.

FIG. 1C illustrates a prior art aromatherapy water pipe 700 having an outside surface 700a, as is known to those of skill in the art and having an inhalation aperture 701 from which a user inhales and a percolator assembly 703 for being immersed in water and an input port 702, whereby when using such a prior art aromatherapy water pipe 700, a bowl (not shown) is placed on the input port 702 and the user places phyto material (not shown) within the bowl (not shown) for then being combusted by a flame source and caused to smoke, whereby upon inhalation from the inhalation aperture 701 by the user, the smoke and ambient air 555 flow into the input port 702 and into the water via the percolator assembly 703 for being somewhat purified by the water for then being inhaled from the inhalation aperture 701 by the user.

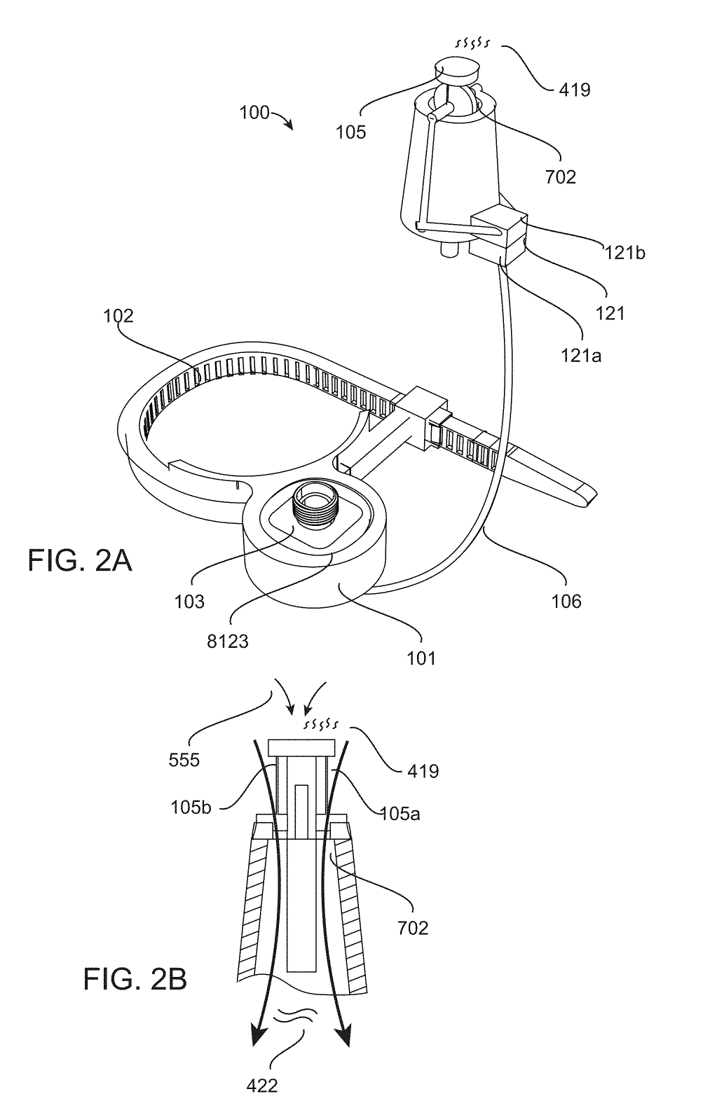

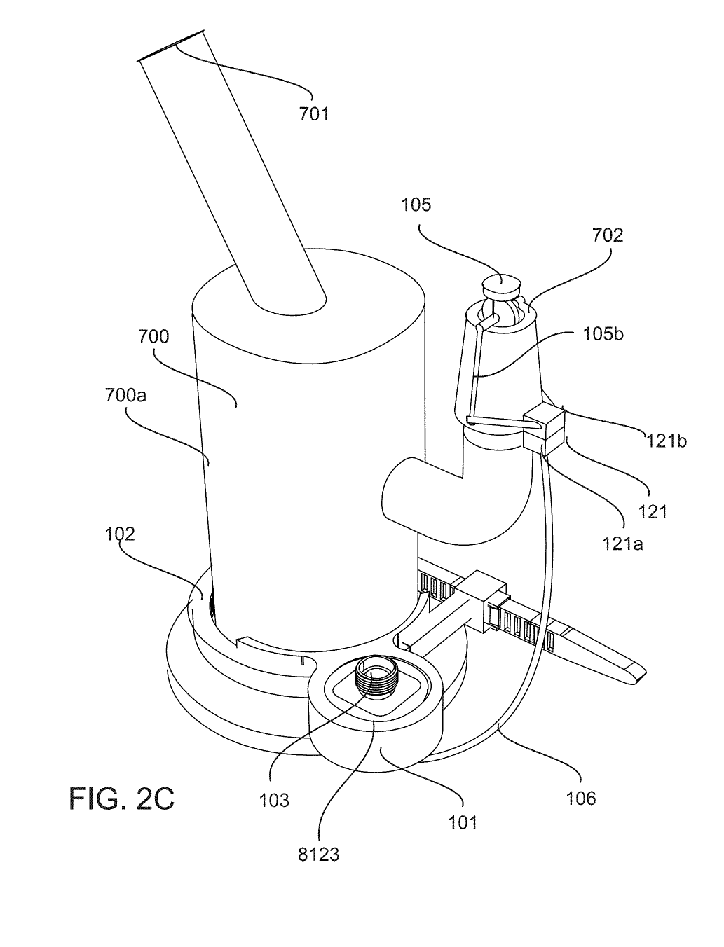

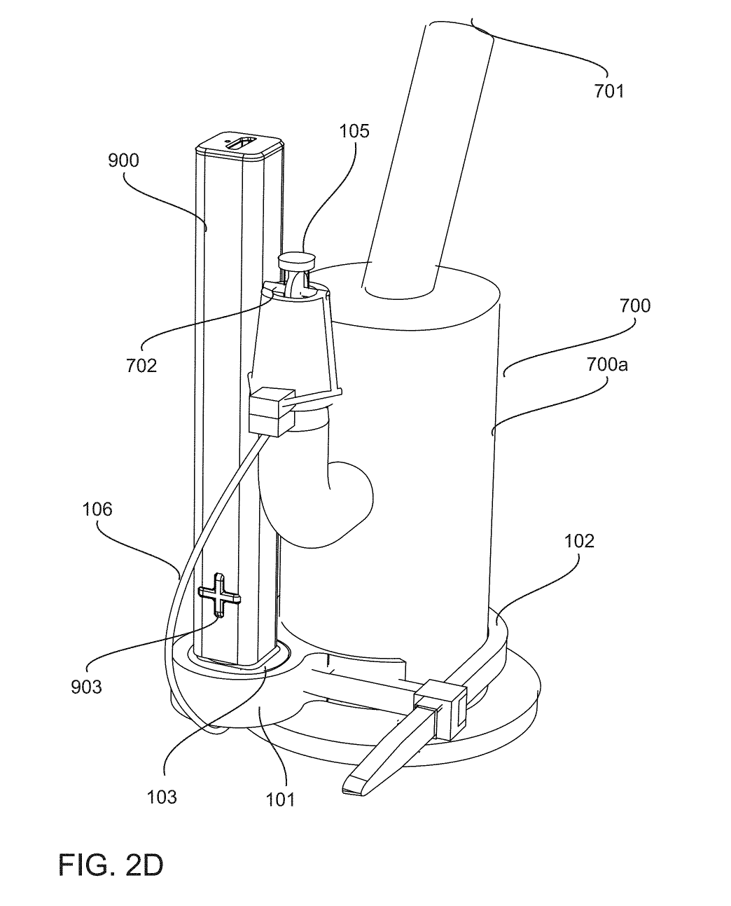

FIG. 2A illustrates the preferred embodiment of the invention a phyto material vaporization device 100 for being powered by an atomizer power supply 900 having a central axis 920 and a control button 903 for being attached to an aromatherapy water pipe 700 (not shown in this figure, but shown in FIG. 2C) having an input port 702 in fluid communication with an inhalation aperture 701 comprising a water pipe engagement member 101 for being releasably coupled with the aromatherapy water pipe 700. A fastening mechanism 102 for releasably fastening the water pipe engagement member 101 to the aromatherapy water pipe 700.

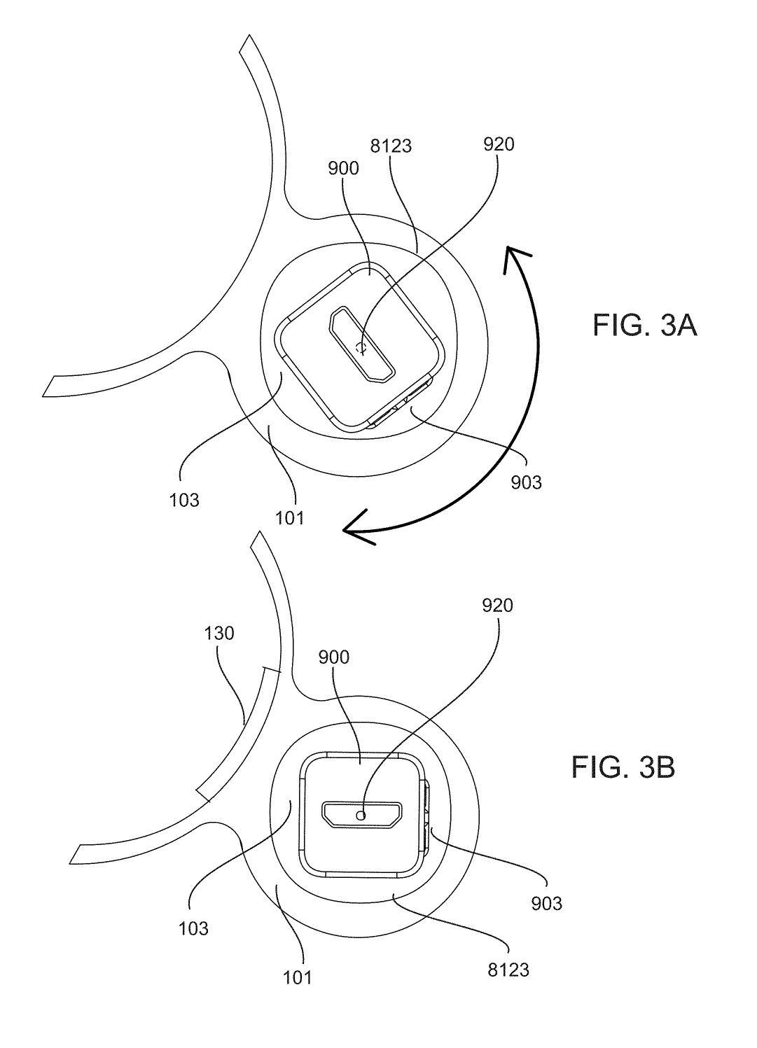

An atomizer power supply receptacle 103 for releasably coupling with the atomizer power supply 900 for receiving of electrical energy therefrom, the atomizer power supply receptacle 103 for being frictionally engaged within the water pipe engagement member 101 through a frictional engagement zone 8123 for allowing of rotation of the atomizer power supply receptacle 103 within the water pipe engagement member 101 about the central axis 920 of the atomizer power supply 900. Having the rotation of the atomizer power supply receptacle 103 within the water pipe engagement member 101 advantageously allows for the atomizer power supply 900 to be oriented in a manner that enables comfortable use of the atomizer power supply 900 by the end user (FIGS. 3A and 3B).

A conduction heating element 105 for receiving of the phyto material 419 and for being disposed proximate the aromatherapy water pipe input port 702 having an electrical connection 706 for electrically coupling with the atomizer power supply receptacle 103, wherein, upon coupling of the atomizer power supply 900 with the atomizer power supply receptacle 103 and by pushing of the control button 903, electrical energy from the atomizer power supply 900 flows to the conduction heating element 105 for heating of the phyto material 419 which then forms into phyto material vapor 421 and together with ambient air 555 (FIG. 2B) flows into the input port 702 upon inhalation from the inhalation aperture 701 by the user.

FIG. 2B illustrates a portion of the preferred embodiment of the invention 100 with the conduction heating element 105 and the first and second electrical contacts 105a and 105b. When the conduction heating element 105 is heated and phyto material 419 is applied thereof, the phyto material 419 is vaporized and vapor emitted therefrom as well as ambient air 555 flows into the input port 702 upon inhalation from the inhalation aperture 701 by the user. The conduction heating element 105 is disposed within proximity to the input port 702 an restricts airflow entering therein. Furthermore vapor 422 and ambient air 555 flow past the conduction heating element 105 approximately parallel to first and second electrical contacts 105a and 105b and into the input port 702 of the water pipe 700.

FIG. 2C illustrates the preferred embodiment of the invention 100 coupled to the aromatherapy water pipe 700 with the atomizer power supply 900 not shown for clarity. The conduction heating element 105 and the first and second electrical contacts 105a and 105b are shown as well as the releasable electrical coupling 212 with the first portion 121a electrically coupled with the atomizer power supply receptacle 103 and the second portion 121b electrically coupled with the conduction heating element 105. Optionally the releasable electrical coupling 212 comprises a magnet therein.

FIG. 2D illustrates the preferred embodiment of the invention 100 coupled to the aromatherapy water pipe 700 and to the outside surface 700a thereof with the atomizer power supply 900 shown. The conduction heating element 105 is disposed proximate the input port 702 and the control button 903 oriented such that it is easily accessible to the user. Referring to FIGS. 3A and 3B, the a frictional engagement zone 8123 allows for rotating of the atomizer power supply 900 after the atomizer power supply receptacle 103 and the power output port 905 have been frictionally engaged. In the case when a 510 thread is used between the atomizer power supply receptacle 103 and the power output port 905, or in the case of another threaded coupling, the threads are cut at different orientations and as such when atomizer power supply receptacle 103 and the power output port 905 are frictionally engaged with each other, the control button 903 may be oriented so that it is against the aromatherapy water pipe 900 and is not easily accessible. Therefore having the possibility to rotate of the atomizer power supply receptacle 103 about the central axis 920 of the atomizer power supply 900 is advantageous.

Advantageously the atomizer power supply receptacle 103 is frictionally engaged within the water pipe engagement member 101 for allowing of rotation of the atomizer power supply receptacle 103 within the water pipe engagement member 101 about a central axis 920 of the atomizer power supply 900. Rotating allows for the control button 903 to be placed in a convenient orientation for the user (FIGS. 3A and 3B) as the thread is not always cut identically between manufactured parts and if the thread was fixed in place then the control button may be facing the water pipe 700 and may therefor not be orientated conveniently for the user.

Advantageously this places the button of the atomizer power supply 900 so that it can easily be triggered using the fingers of the user. It allows for an existing prior art water pipe 700 to be used as an electric water pipe and thus eliminating the need for torches.

Advantageously, the embodiments of the invention conveniently fit on to many existing aromatherapy water pipes 700 and do not necessarily need to be sold with one. This allows for a much more compact shipping packaging as well as its not as fragile because the water pipe is not included in the shipment. It also reduces the shipping weight.

There area also many existing atomizer power supplies on the market that have the 510 or eGo threads, as such being able to use these again and to recycle them is advantageous as it reduces E waste and gives them another purpose when considering the embodiments of the invention.

Advantageously the aromatherapy water pipe engagement member comprises a soft thermoplastic urethane or rubber materials for it to deform to various diameter water pipes and to contour to their outside surface.

Numerous other embodiments are envisaged without departing from the spirit or scope of the invention.

What I claim is:

1. A phyto material vaporization device for being powered by an atomizer power supply having a central axis and a control button for being attached to an aromatherapy water pipe having an input port in fluid communication with an inhalation aperture comprising:
a water pipe engagement member for being releasably coupled with the aromatherapy water pipe;
a fastening mechanism for releasably fastening the water pipe engagement member to the aromatherapy water pipe;
an atomizer power supply receptacle for releasably coupling with the atomizer power supply for receiving of electrical energy therefrom;
a conduction heating element for receiving of the phyto material and for being disposed proximate the aromatherapy water pipe input port for restricting of ambient air flowing into the input port and having an electrical connection for electrically coupling with the atomizer power supply receptacle, wherein, upon coupling of the atomizer power supply with the atomizer power supply receptacle and by actuating of the control button, electrical energy from the atomizer power supply flows to the conduction heating element for heating of the phyto material for forming phyto material vapor and for the formed phyto material vapor and ambient air to flow into the input port and past the conduction heating element upon inhalation from the inhalation aperture by the user.

2. A phyto material vaporization device according to claim 1 wherein the fastening mechanism comprises an adhesive disposed on the water pipe engagement member for adhering to an outside surface of the aromatherapy water pipe.

3. A phyto material vaporization device according to claim 1 wherein the fastening mechanism comprises a zip tie for upon tightening for frictionally engaging the water pipe engagement member to an outside surface of the aromatherapy water pipe.

4. A phyto material vaporization device according to claim 1 wherein the electrical connection for electrically coupling with the atomizer power supply receptacle comprises: a releasable electrical coupling comprising a first portion electrically coupled with the atomizer power supply receptacle and a second portion electrically coupled with the conduction heating element.

5. A phyto material vaporization device according to claim 1 wherein the water pipe engagement member comprises one of a soft thermoplastic urethane and a rubber material.

6. A phyto material vaporization device according to claim 1 wherein the conduction heating element comprises ceramic substrate having a resistive material applied in order to generate heat when the electrical energy from the atomizer power supply flows to the conduction heating element.

7. A phyto material vaporization device according to claim 6 wherein the conduction heating element comprises first and second electrical contacts and, wherein a resistive and conductive material is applied between these contacts and results in the heating element to heat up to degrees Fahrenheit and upon the application of phyto material to the conduction heating element, the phyto material forms phyto material vapor and the formed phyto material vapor and ambient air flow into the input port approximately parallel to these electrical contacts and past the conduction heating element upon inhalation from the inhalation aperture by the user.

8. A phyto material vaporization device according to claim 1 wherein the atomizer power supply receptacle comprises a thread for releasably coupling to the atomizer power supply.

9. A phyto material vaporization device according to claim 1 comprising: a frictional engagement zone disposed between the atomizer power supply receptacle and the water pipe engagement member, wherein the frictional engagement zone allows for rotation of the atomizer power supply receptacle within the water pipe engagement member about a central axis of the atomizer power supply.

10. A phyto material vaporization device for being powered by an atomizer power supply having a central axis and a control button for being attached to an aromatherapy water pipe having an input port in fluid communication with an inhalation aperture comprising:
a water pipe engagement member for being releasably coupled with the aromatherapy water pipe;
a fastening mechanism for releasably fastening the water pipe engagement member to the aromatherapy water pipe;
an atomizer power supply receptacle for releasably coupling with the atomizer power supply for receiving of electrical energy therefrom, the atomizer power supply receptacle for being frictionally engaged within the water pipe engagement member for allowing of rotation of the atomizer power supply receptacle within the water pipe engagement member about a central axis of the atomizer power supply;
a conduction heating element for receiving of the phyto material and for being disposed proximate the aromatherapy water pipe input port having an electrical connection for electrically coupling with the atomizer power supply receptacle, wherein, upon coupling of the atomizer power supply with the atomizer power supply receptacle and by pushing of the control button, electrical energy from the atomizer power supply flows to the conduction heating element for heating of the phyto material for forming phyto material vapor and for the formed phyto material vapor and ambient air to flow into the input port and past the conduction heating element upon inhalation from the inhalation aperture by the user.

11. A phyto material vaporization device for being powered by an atomizer power supply having a central axis and a control button for being attached to an aromatherapy water pipe having an input port in fluid communication with an inhalation aperture comprising:
a water pipe engagement member for being releasably coupled with the aromatherapy water pipe;
a fastening mechanism for releasably fastening the water pipe engagement member to the aromatherapy water pipe;
an atomizer power supply receptacle for releasably coupling with the atomizer power supply for receiving of electrical energy therefrom;
a conduction heating element comprising first and second electrical contacts and, conduction heating element for receiving of the phyto material and for being disposed proximate the aromatherapy water pipe input port for restricting of ambient air flowing into the input port and having an electrical connection for electrically coupling with the atomizer power supply receptacle, wherein, upon coupling of the atomizer power supply with the atomizer power supply receptacle and by actuating of the control button, electrical energy from the atomizer power supply flows to the conduction heating element for heating of the phyto material for forming phyto material vapor and for the formed phyto material vapor and ambient air to flow into the input port and past the conduction heating element and parallel to the first and second electrical contacts and for subsequent inhalation from the inhalation aperture by the user.
